(19) United States
(12) Patent Application Publication
(10) Pub. No.: US 2020/0140890 A1
(43) Pub. Date: May 7, 2020

(54) PYRALID MOTH EGG, PRODUCING METHOD THEREOF, AND METHOD FOR PRODUCING RECOMBINANT PROTEIN BY USING PYRALID MOTH EGG

(71) Applicants: Jeongbae JEON, Gyeonggi-do (KR); ENBLOC CELL LLC, Gyeonggi-do (KR)

(72) Inventor: Jeongbae JEON, Gyeonggi-do (KR)

(21) Appl. No.: 16/619,137

(22) PCT Filed: Dec. 19, 2017
(86) PCT No.: PCT/KR2017/014988

§ 371 Date: Dec. 4, 2019

(30) Foreign Application Priority Data
Jun. 8, 2017 (KR) 10-2017-0071919

(51) Int. Cl. C12N 15/86; A01K 67/033

(52) U.S. Cl. CPC C12N 15/86; A01K 67/0339; C12N 2710/14041; A01K 2227/706; A01K 2267/01; A01K 2217/05

(57) ABSTRACT

The present invention relates to means and methods for increasing the efficiency of recombinant protein expression and, more particularly, to means and methods for optimizing the industrial production of recombinant proteins in pyralid moths eggs (which are insect pests of stored foods), especially Mediterranean flour moth eggs (Ephestia kuehniella). Furthermore, the present invention relates to the pyralid moths eggs itself which contains a recombinant baculovirus and, infection of recombinant baculovirus into pyralid moths eggs and, transformation or transduction or transfection by recombinant baculoviruses or bacmids. In addition, it relates to an appropriate device for carrying out the method of the present invention.

### TECHNICAL FIELD

The present invention can be included in the field of biotechnology and is intended to increase the efficiency of recombinant protein expression. And more particularly to means and methods involving optimization of the industrial production of recombinant proteins in pyralid moths eggs (especially Ephestia kuehniella eggs) which are insect pests of stored foods. Furthermore, the present invention is not limited to pyralid moths eggs themselves containing recombinant baculoviruses, relates to infection of recombinant baculovirus into pyralid moths eggs, and relates to transformation or transduction or transfection by recombinant baculoviruses or bacmids.

### BACKGROUND

Recombinant protein expression systems used as vaccines, therapeutic molecules or diagnostic reagents utilize a variety of expression vectors, and It has been developed based on bioreactors using microbial culture technology such as Escherichia coli (which is a bacteria or prokaryotes) and yeast (which is a eukaryotes), and also, It has been developed based on bioreactors using plant cell culture technology, animal cell culture technology, insect cell culture technology.

In particular, baculovirus expression vector system (BEVS), which has been developed since the 1980s, is known to be the most efficient method for producing all-purpose recombinant proteins because of its high over-expression potential and rapid development speed. Currently, recombinant baculovirus vectors most commonly used in industry for expression of recombinant proteins are based on Autographa californica multinuclear polyhedrosis virus (AcMNPV) and the insect cells which are Spodoptera frugiperda 9 (Sf9) or 21 (Sf21) as appropriate expression hosts. Since the development of BEVS, hundreds of recombinant proteins, from cytosolic enzymes to membrane-bound proteins, have been successfully produced through recombinant baculovirus-infected insect cells.

However, a recombinant protein expression system based on a stainless steel stirred-tank bioreactor requires several years of time and high cost to build up, and requires a highly skilled workforce for operation of the system, and has a problem of low reliability because it is easy to pollute and it is difficult to verify the expression results. Since the 1990s, using the wave bioreactor made of a disposable plastic bag, the above problem can be solved in scale-up production stage. However, in the final stage of mass production, they are faced with problems of inefficiency, high cost, technical complexity and limited scalability, and it becomes a technical and economic barrier to producing new or existing recombinant proteins.

Recently used medicine for Ebola Virus Disease (EVD), a mixture of three kinds of monoclonal antibodies, did not use plant cell culture technology based on bioreactors. It is produced through transfection to live tobacco plants (Nicotiana tabacum), but is generally not suitable for mass production of recombinant proteins, because it has problems the relatively low productivity and the tobacco production cycle that takes several months. However, insects can grow in size about 5000 times within two weeks because of the fast metabolism, and insects are known to be the most efficient protein producers, because silk gland cell of Silkworm (Bombyx mori) belonging to Bombycidae can produce about 80 micrograms protein/cell/day, but mammalian cell culture systems may produces only about 50 picograms protein/cell/day. As living biofactories, insects are therefore a promising alternative to traditional bioreactor based microbial culture and cell culture technologies due to the flexibility, scalability, and automation potential for mass production, and it has efficiency of development and rapid development speed. It also calls the name as bioreactor using live insects.

Larvae and pupae of Lepidopteran insects such as Silkworm and Cabbage looper (Trichoplusia ni) belonging to Noctuidae are most commonly used as ‘bioreactor using live insects’, and it mainly uses AcMNPV or Bombyx mori nuclear polyhedrosis virus (BmNPV) vectors. The insect larvae and pupae body fluids are aseptic and contain protease inhibitors (Pls) so that protein degradation is not likely to occur, and thus the substances expressed by BEVS can be stably present. In the case of the cell culture method, the cell culture medium contains fetal bovine serum (FBS), which requires a complicated biochemical purification method for the isolation of the target recombinant protein, but it can be recovered relatively easily in insect body fluids. In particular, AcMNPV and BmNPV are capable of expressing foreign genes in almost all cells except for some tissues in insect body, and their recombinant protein productivity is remarkably higher than that of cell culture methods. In particular, according to KR10-0921812, the cellulase specific activity of Sf9 insect cell culture method showed an expression efficiency of 15.25 units (U) per 1 ml, whereas was about 226 times of expression efficiency as silkworm larvae showed 3439 units per 1 ml.

Methods for inoculation of recombinant baculovirus against insects used as ‘bioreactor using live insects’ include a method in which recombinant baculovirus is directly injected into a body of larva or pupa by a syringe (injection), a method of oral inoculation in which recombinant baculovirus is mixed with feed of larvae to infect through the gastrointestinal mucosa by feeding, a soaking method in which a larva or a pupa is soaked into a liquid containing a recombinant baculovirus and is infected through a gastrointestinal mucosa and a respiratory tract are known. Comparisons in silkworm moths have shown that most proteins showed the highest expression rate in larvae than in pupae, furthermore, it is known that the susceptibility to recombinant baculovirus infection decreases as the age of silkworm moth pupa increases. In addition, since the silkworm moth pupa cannot use the oral inoculation method, it is tedious and time-consuming to manually perform the injection method, and the thick cocoon covered in the pupa should also be removed manually before the recombinant baculovirus inoculation. Due to the reduced protein expression generally from the silkworm moth pupa and the difficulty of manipulation, the bioreactor using live insects is generally used in the scale up stage rather than in the mass production stage, and it is carried out through the oral inoculation method to the larvae rather than the injection method or soaking method to the larvae and pupa.

Recently, in WO2012/168493, WO2012/168492, and WO2017/046415, a new recombinant baculovirus vector, a Cabbage looper pupa, and an automated system using a machine capable of continuously injecting recombinant baculovirus into a pupa of about 3000 per hour has been described. However, until the present, the larvae of the silkworm moth, Noctuidae, Saturniidae, and Sphingidae, which have been mainly used as bioreactor using live insects, feed on fresh leaves of specialized host plants, or artificial feeds that replace it are also made using expensive materials. In addition, because of the relatively big larval size, as instar larval stage increases, individual breeding is required, there is a problem of ensuring sufficient breeding space and excessive labor input, and most of the pupa is covered with thick cocoons. Due to all these disadvantages, lepidopteran insects belonging to the silkworm moth, Noctuidae, Saturniidae, and Sphingidae are not suitable for industrial mass rearing, and the recombinant protein expression system using them is mainly utilized in the scale up stage for production. Eventually, in order to solve the problems of inefficiency, high cost, technical complexity and limited scalability by completely replacing the stainless steel stirred-tank bioreactor until the final stage of mass production, the selection of new bioreactor using live insects suitable for industrial mass rearing and the development of new recombinant protein expression systems that can automate the inoculation of recombinant baculoviruses is required.

In the insect industry all over the world, large quantities of pyralid moths are being mass reared for the purpose of producing commercial natural enemies, and pyralid moths eggs (which are insect pests of stored foods) are used on a large scale for an alternative host of parasites such as Trichogramma spp. and substitute diets of predators such as Orius spp. In particular, Almond moth (Cadra cautella) or Mediterranean flour moth (Ephestia kuehniella) of material cost is low, because by-products such as rice bran from the rice processing process and wheat bran from the wheat processing process are used, the length of final instar larval stage larvae is about 10˜24 mm, so its size is small, and because there is no cannibalism, group rearing is possible. In addition, number of eggs laid are about 100˜700 per a female, so its quantity is a lot, the length of major axis from eggs are 0.5˜1.0 mm, and because eggs have characteristics that are scattered individually, and it is possible to easily separate the pyralid moths eggs and harvest it in large quantities through the egg collection device (described as an adult emergence device or egg collection box) as in the embodiment of KR10-1053217 or KR10-1053217.

However, all insect eggs are surrounded by a multi-layered structure eggshell (vitelline membrane, waxy layer, and chorionic layer) with outside the cell membrane, through this, respiration is possible, but it prevents infection of viruses and bacteria from the outside. Eventually, though pyralid moths eggs (which are insect pests of stored foods) such as Mediterranean flour moth are being produced in large quantities all over the world, however, since the inoculation of recombinant baculovirus is very difficult, until the present, it has not been used as an bioreactor using live insects for the expression of recombinant proteins. On the other hand, according to WO2010/081078, for the purpose of observing the embryogenesis process in eggs of Drosophila spp. which widely used as biotechnology materials, a method of removing the chorionic layer and wax layer of the eggshell without toxicity using the embryo permeabilization solvent (EPS; consisting of 90% D-limonene+5% Cocamide DEA+5% ethoxylated alcohol) and 3% sodium hypochlorite has been described. However, bioreactor using live insects that is suitable for mass rearing and can almost completely automate the inoculation of recombinant baculovirus, it can reduce costs associated with recombinant protein expression and increase efficiency from the scale up production stage to the final mass production stage in industrial scale, it has not been reported yet.

### Technical Solution

The present invention relates to means and methods for increasing the efficiency of recombinant protein expression and, more particularly, to means and methods for optimizing the industrial production of recombinant proteins in pyralid moths eggs (which are insect pests of stored foods), especially Mediterranean flour moth eggs (Ephestia kuehniella). Furthermore, the present invention be applied not only to the pyralid moths eggs itself which contains a recombinant baculovirus, infection of recombinant baculovirus into pyralid moths eggs, transformation or transduction or transfection by recombinant baculoviruses or bacmids. In addition, it relates to an appropriate device for carrying out the method of the present invention.

The recombinant protein of the present invention is preferably selected from the group consisting of subunit monomeric vaccine, subunit multimeric vaccine, virus like particle, therapeutic protein, antibody, enzyme, cytokine, blood clotting factor, anticoagulant, receptor, hormone, diagnostic protein reagents, and the green fluorescent protein (GFP).

In the present invention, which one are selected from the group consisting of Meal moth (Pyralis farinalis), Stored nut moth (Paralipsa gularis), Indianmeal moth (Plodia interpunctella), Tea Tabby (Aglossa dimidiatus), Rice moth (Corcyra cephalonica), Mediterranean flour moth (Ephestia kuehniella), Cacao moth (Ephestia elutella), and Almond moth (Cadra cautella) and, after mass rearing insects belonging to the pyralid moths (which are insect pests of stored foods), pyralid moths eggs separated through egg collection device and it supplied in large quantities and, preferably, pyralid moths eggs belonging to the species Mediterranean flour moth is mass produced and it supplied.

And above-mentioned pyralid moths eggs surrounded by an eggshell having a multilayer structure of a vitelline membrane, a waxy layer and a chorionic layer is treated with a nontoxic chemical to remove chorionic layer of the outermost layer, preferably treated with 3% sodium hypochlorite to remove chorionic layer. In addition, above-mentioned pyralid moths eggs (chorionic layer is removed) is treated with a nontoxic composition to remove wax layer, preferably treated with embryo permeabilization solvent (EPS; consisting of 90% D-limonene+5% Cocamide DEA+5% ethoxylated alcohol) to remove wax layer.

And the chorionic layer and wax layer of eggshell are removed and the surface-disinfected pyralid moths eggs and, after being packaged in a sterile container that prevents contamination from the outside but has a ventilation structure for breathing and internal gas exchange and, it is stored at low temperature condition of 4˜8° C., preferably, in the species Mediterranean flour moth, pyralid moths eggs (chorionic layer and wax layer of eggshell are removed) are stored in a sterilized container with a ventilation structure at low temperature condition of 4˜8° C. At above-mentioned low temperature condition of about 4˜8° C., pyralid moths eggs can be stored for up to about 4 weeks, it is possible to supply to all over the world through refrigerated transport using aircraft.

And the recombinant baculovirus (derived from Autographa californica multinuclear polyhedrosis virus; AcMNPV) is sprayed onto the above-mentioned pyralid moths eggs or, preferably, inoculated the recombinant baculovirus derived from AcMNPV by use soaking method the above-mentioned pyralid moths eggs in the liquid containing the recombinant baculovirus derived from AcMNPV. Also, incubate above-mentioned pyralid moths eggs (inoculated the recombinant baculovirus derived from AcMNPV) for a time sufficient to express at least one recombinant protein.

And cultured for a time sufficient to express the recombinant protein and, collect above-mentioned pyralid moths eggs comprising at least one recombinant protein and, store frozen at −20° C. condition. At above-mentioned −20° C. frozen condition, pyralid moths eggs can be stored for up to about one year, it is possible to supply to all over the world through the process of refrigeration transportation using aircraft and ship.

Finally, one or more recombinant proteins are included and crushes the above-mentioned pyralid moths eggs stored in the frozen. And from crushed liquids of above-mentioned pyralid moths eggs comprising at least one recombinant protein is isolated and purified to produce the final recombinant protein product.

### Advantageous Effects

The recombinant protein expression system based on the novel bioreactor using live insects proposed in the present invention is suitable for industrial mass rearing and can automate the inoculation of recombinant baculovirus. It can solve the problems of inefficiency, high cost, technical complexity, limited scalability in the production of recombinant proteins based on existing baculovirus expression vector system (BEVS) and bioreactor and insect cell culture technology. It compared with insect cell culture technology based on existing bioreactors, recombinant protein expression efficiency can be improved from 5 times (WO2017/046415) to 226 times (KR10-0921812).

It is also possible to replace the wave bioreactor in the scale up production stage as well as the stainless steel stirred-tank bioreactor in the final production stage, and it can reduce costs associated with recombinant protein expression and increase efficiency.

### BRIEF DESCRIPTION OF THE DRAWINGS

FIG. 1 is a diagram showing a whole process for producing a recombinant protein by inoculating a recombinant baculovirus vector derived from AcMNPV to the pyralid moths eggs (which are insect pests of stored foods) according to an embodiment of the present invention.

### DESCRIPTION OF THE REFERENCE NUMERALS IN THE DRAWINGS

1: Process of removing the chorionic layer and wax layer from eggshell of pyralid moths eggs
2: Process of inoculation by spraying or soaking recombinant baculovirus into pyralid moths eggs, and process of expression a recombinant protein
3: Process of producing products by harvesting and purifying recombinant proteins from pyralid moths eggs

### DETAILED DESCRIPTION

Hereinafter, preferred embodiments of the present invention will be described in detail with reference to the accompanying drawings so that those skilled in the art can easily carry out the present invention. In the following detailed description of the preferred embodiments of the present invention, a detailed description of known functions and configurations incorporated herein will be omitted when it may make the subject matter of the present invention rather unclear. The same reference numerals are used throughout the drawings for portions having similar functions and functions.

Incidentally, “including” an element means that it may include other elements, not excluding other elements unless specifically stated otherwise.

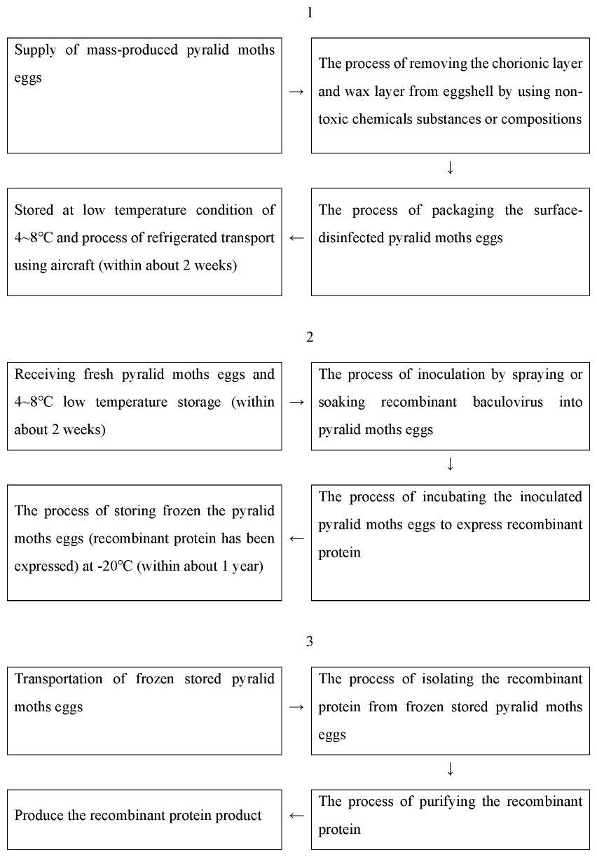

FIG. 1 is a diagram showing a whole process for producing a recombinant protein by inoculating a recombinant baculovirus vector derived from AcMNPV to the pyralid moths eggs according to an embodiment of the present invention. As shown in FIG. 1, the production of recombinant proteins in the pyralid moths eggs according to the embodiment of the present invention may be composed of the following processes. The process of removing the chorionic layer and wax layer from eggshell of pyralid moths eggs (1), process of inoculation by spraying or soaking recombinant baculovirus into pyralid moths eggs, and process of expression a recombinant protein (2), process of producing products by harvesting and purifying recombinant proteins from pyralid moths eggs (3).

The process (1) of removing the chorionic layer and wax layer from eggshell of pyralid moths eggs of the present invention, which one are selected from the group consisting of Meal moth (Pyralis farinalis), Stored nut moth (Paralipsa gularis), Indianmeal moth (Plodia interpunctella), Tea Tabby (Aglossa dimidiatus), Rice moth (Corcyra cephalonica), Mediterranean flour moth (Ephestia kuehniella), Cacao moth (Ephestia elutella), and Almond moth (Cadra cautella) and, after mass rearing insects belonging to the pyralid moths (which are insect pests of stored foods), pyralid moths eggs separated through egg collection device and it supplied in large quantities and, preferably, pyralid moths eggs belonging to the species Mediterranean flour moth is mass produced and it supplied.

And above-mentioned pyralid moths eggs surrounded by an eggshell having a multilayer structure of a vitelline membrane, a waxy layer and a chorionic layer is treated with a nontoxic chemical to remove chorionic layer of the outermost layer, preferably treated with 3% sodium hypochlorite to remove chorionic layer. In addition, above-mentioned pyralid moths eggs (chorionic layer is removed) is treated with a nontoxic composition to remove wax layer, preferably treated with embryo permeabilization solvent (EPS; consisting of 90% D-limonene+5% Cocamide DEA+5% ethoxylated alcohol) to remove wax layer.

Finally, the chorionic layer and wax layer of eggshell are removed and the surface-disinfected pyralid moths eggs and, after being packaged in a sterile container that prevents contamination from the outside but has a ventilation structure for breathing and internal gas exchange and, it is stored at low temperature condition of 4˜8° C., preferably, in the species Mediterranean flour moth, pyralid moths eggs (chorionic layer and wax layer of eggshell are removed) are stored in a sterilized container with a ventilation structure at low temperature condition of 4˜8° C. At above-mentioned low temperature condition of about 4˜8° C., pyralid moths eggs can be stored for up to about 4 weeks, it is possible to supply to all over the world through refrigerated transport using aircraft.

The recombinant protein of the present invention is preferably selected from the group consisting of subunit monomeric vaccine, subunit multimeric vaccine, virus like particle, therapeutic protein, antibody, enzyme, cytokine, blood clotting factor, anticoagulant, receptor, hormone, diagnostic protein reagents, and the green fluorescent protein (GFP).

In a preferred embodiment, the recombinant protein is a virus-like particle protein, which is preferably selected from the group consisting of:

(a) Porcine circo virus capsid protein, preferably from porcine circovirus type 2 (e.g., SEQ ID NO.: 26),

(b) Foot and mouth disease virus VP1, VP3 or VP0 protein,

(c) Canine parvovirus VP1 and VP2 proteins,

(d) Porcine parvovirus VP1 and VP2 proteins,

(e) Human norovirus (genogroup I or II) capsid protein,

(f) Calicivirus capsid protein,

(g) Human papillomavirus L1 protein, preferably from human papillornavirus 16,

(h) Hepatitis E protein E2,

(i) Infectious bursal disease virus VP1, VP2 and VP3 proteins,

(j) Astrovirus ORF2-encoded proteins,

(k) Influenza virus HA (e.g., SEQ ID NO.: 30), NA and M1 proteins,

(1) Hepatitis B core and surface antigens,

(m) Rabbit calici virus VP60 protein, preferably from rabbit haemorrhagic disease viruses RHDVb and RHDVG1 (e.g., SEQ ID NOs.: 32 and 33),

(n) Human parvovirus VP1 and VP2 proteins

For instance, the recombinant protein may be:

Porcine circovirus capsid protein, preferably from porcine circovirus type 2, which is, for example, represented by the amino acid sequence of SEQ ID NO.: 26 or encoded by the nucleic acid sequence of SEQ ID NO.: 25.

Foot and mouth disease virus (FMDV) VP1, VP3 and VP0 protein, the sequence of which is indicated or can be derived, for example, from the following sequences:
FMDV serotype 0 complete genome: GenBank JX570650.1
FMDV serotype A complete genome: GenBank HQ832S92.1
FMDV serotype C complete genome: GenBank AYS93810.1
FMDV serotype SAT 1 complete genome: GenBank AY593846.1
FMDV serotype SAT 2 complete genome: GenBank JX014256.1
FMDV serotype ASIA 1 complete genome: GenBank HQ631363.1

Canine parvovirus VP1 and VP2 protein, the sequence of which is indicated or can be derived, for example, from the following sequences:
Canine parvovirus VP1 gene for capsid protein VP1, partial cds. strain 1887/f/3: GenBank AB437434.1
Canine parvovirus VP1 gene for capsid protein VP1, partial cds. strain 1887/M/2: GenBank AB437433.1
Canine parvovirus VP2 gene, complete cds. strain HNI-2-13: GenBank AB120724.1
Canine parvovirus VP2 gene, complete cds. strain HNI-3-4: GenBank AB120725.1
Canine parvovirus VP2 gene, complete cds. strain HNI-3-11: GenBank AB120726.1
Canine parvovirus VP2 gene, complete cds. strain HNI-4-1: GenBank AB120727.1
Canine parvovirus VP2 gene, complete cds. strain HNI-1-18: GenBank AB120728.1
Canine parvovirus VP2 protein (VP2) gene, complete cds.: GenBank DQ354068.1
Canine parvovirus VP2 gene, complete cds. strain HCM-6: GenBank AB120720.1
Canine parvovirus isolate Taichung VP2 gene, complete cds.: GenBank AY869724.1
Canine parvovirus VP2 gene, complete cds. strain HCM-8: GenBank AB120721.1
Canine parvovirus type 1 proteins VP1 and VP2: GenBank AB518883.1
Canine parvovirus type 2a VP1 and VP2: GenBank M24003.1
Canine parvovirus type 2b VP2: GenBank FJ005265.1
Canine parvovirus Type 2c VP2: GenBank FJ005248.1

Porcine parvovirus VP1 and VP2 protein, the sequence of which is indicated or can be derived, for example, from the following sequences:
Porcine parvovirus strain 693a: GenBank JN400519.1
Porcine parvovirus strain 8a: GenBank JN400517.1

Human parvovirus VP1 and VP2 protein, the sequence of which is indicated or can be derived, for example, from the following sequences:
Human parvovirus B19 VP1 complete cds.: GenBank AF264149.1
Human parvovirus B19 isolate Vn115 NS1 (NS1), 7.5 kDa protein (NS1), VP1 (VP1), 9.5 kDa protein (VP1), and VP2 (VP2) genes, complete cds.: GenBank DQ357065.1
B19 virus isolate FoBe VP1 (VP1) and VP2 (VP2) genes, complete cds.: GenBank AY768535.1
B19 virus isolate Br543 NS1 (NS1), VP1 (VP1), and VP2 (VP2) genes, complete cds.: GenBank AY647977.1
Human parvovirus 4 isolate VES065CSF NS1, VP1, and VP2 genes, complete cds.: GenBank HQ593532.1
Human parvovirus 4 isolate VES085CSF NS1 gene, partial cds. and VP1 and VP2 genes, complete cds.: GenBank HQ593531.1
Human parvovirus B19 strain BB-2 NS1, VP1, and VP2 genes, complete cds.: GenBank KF724387.1

Human norovirus (genogroup I or II) capsid protein, the sequence of which is indicated or can be derived, for example, from the following sequences:
Norwalk virus: GenBank M87661, NP056821
Southampton virus: GenBank L07418
Mexico virus: GenBank U22498
Seto virus: GenBank AB031013
Chiba virus: GenBank AB042808
Lordsdale virus: GenBank X86SS7
Snow Mountain virus: GenBank U70059
Hawaii virus: GenBank U07611

Rabbit haemorrhagic disease virus VP60 protein, the sequence of which is indicated or can be derived, for example, from the following sequence:
RHDV AST/89 complete genome: GenBank Z49271.2
RHDV N11 complete genome: GenBank KM878681.1
RHDV CBVali6 complete genome: GenBank KM979445.1
SEQ ID NO.: 32
SEQ ID NO.: 33

Human papillomavirus L1 protein, the sequence of which is indicated or can be derived, for example, from the following sequences:
HPV 6: GenBank JN252323.1
HPV 11: GenBank JQ773411.1
HPV 16: GenBank DQ155283.1
HPV 18: GenBank FJ528600.1

Hepatitis E virus E2 protein, the sequence of which is indicated or can be derived, for example, from the following sequences:
Hepatitis E virus, complete genome: NCBI Reference Sequence NC001434.1
Swine hepatitis E virus isolate ITFAE11 capsid protein gene: GenBank JN861806.1

Infectious bursal disease virus VP1, VP2 and VP3 proteins, the sequence of which is indicated or can be derived, for example, from the following sequences:
Infectious bursal disease virus VP1 (VP1) gene, complete cds.: GenBank AY099457.1
Infectious bursal disease virus isolate PT VP1 gene, complete cds.: GenBank DQ679814.1
Infectious bursal disease virus isolate OE/G2 VP1 gene, complete cds.: GenBank DQ679813.1
Infectious bursal disease virus isolate OA/G1 VP1 gene, complete cds.: GenBank DQ679812.1
Infectious bursal disease virus isolate HOL VP1 gene, complete cds.: GenBank DQ679811.1
Infectious bursal disease virus strain TL2004 VP1 gene, complete cds.: GenBank DQ118374.1
Infectious bursal disease virus isolate CA-K785 VP1 gene, complete cds.: GenBank JF907705.1
Infectious bursal disease virus isolate D495 VP1 gene, complete cds.: GenBank JF907704.1
Infectious bursal disease virus strain A-BH83 VP1 mRNA, complete cds.: GenBank EU544149.1
Infectious bursal disease virus strain Cro-Pa/98 VP1 gene, complete cds.: GenBank EU184690.1
Infectious bursal disease virus VP2 mRNA, complete cds.: GenBank AY321509.1
Infectious bursal disease virus VP2, VP3, VP4 genes, complete cds.: GenBank M97346.1
Infectious bursal disease virus VP2 gene, complete cds.: GenBank AF508177.1

Calicivirus capsid protein, the sequence of which is indicated or can be derived, for example, from the following sequences:
Feline calicivirus capsid protein gene, complete cds.: GenBank L09719.1
Feline calicivirus capsid protein gene, complete cds.: GenBank L09718.1
Human calicivirus HU/NLV/Wortley/90/UK RNA for capsid protein (ORF2), strain HU/NLV/Wortley/90/UK: GenBank AJ277618.1
Human calicivirus HU/NLV/Thistlehall/90/UK RNA for capsid protein (ORF2), strain HU/NLV/Thistlehall/90/UK: GenBank AJ277621.1
Human calicivirus HU/NLV/Valetta/95/Malta RNA for capsid protein (ORF2), strain HU/NLV/Valetta/95/Malta: GenBank AJ277616.1
Human calicivirus NLV/Stav/95/N or capsid protein gene, complete cds.: GenBank AF145709.1
Bovine enteric calicivirus strain Bo/CV500-OH/2002/US capsid protein gene, complete cds.: GenBank AYS491SS.1
Human calici virus NLV/Mora/97/SE capsid protein gene, complete cds.: GenBank AY081134.1
Human calicivirus NLV/Potsdam 196/2000/DE capsid protein gene, complete cds.: GenBank AF439267.1
Human calicivirus NLV/1581-01/SWE capsid protein gene, complete cds.: GenBank AY247442.1
Human calicivirus Hu/NLV/GII/MD134-10/1987/US capsid protein gene, complete cds.: GenBank AY030313.1

Astrovirus ORF2-encoded proteins, the sequence of which is indicated or can be derived, for example, from the following sequences:
Porcine astrovirus 4 ORF1b gene, partial cds. and ORF2 gene, complete cds.: GenBank JX684071.1
Astrovirus MLB1 HK05, complete genome: NCBI Reference Sequence NC014320.1
Astrovirus wild boar/WBAstV-1/2011/HUN, complete genome: NCBI Reference Sequence NC016896.1
Human astrovirus BF34, complete genome: NCBI Reference Sequence NC024472.1
Astrovirus MLB1 strain Hu/ITA/2007/PR326/MLB1 RNA-dependent RNA polymerase (ORF1b) gene, partial cds. and capsid protein (ORF2) gene, complete cds.: GenBank KF417713.1
Human astrovirus 5 strain Hu/Budapest/HUN5186/2012/HUN nonstructural protein (ORF1a) and nonstructural protein (ORF1b) genes, partial cds. and capsid protein (ORF2) gene, complete cds.: GenBank KF157967.1
Human astrovirus 1 isolate Shanghai capsid protein (ORF2) gene, complete cds.: GenBank FJ792842.1
Human astrovirus type 8 orf2 gene for capsid protein: GenBank Z66541.1

Influenza virus HA, NA and M1 proteins, the sequence of which is indicated or can be derived, for example, from the following sequences:
SEQ ID NO.: 30
Influenza A virus (A/duck/Chiba/25-51-14/2013(H7N1)) HA gene for hemagglutinin, complete cds.: GenBank AB813060.1
Synthetic construct hemagglutinin (HA) mRNA, complete cds.: GenBank DQ868374.1
Influenza virus A (A/swine/Shandong/2/03(H5N1)) hemagglutinin (HA) gene, complete cds.: GenBank AY646424.1
cDNA encoding HA of influenza type A: GenBank E01133.1
Influenza A virus (A/swine/Korea/5452/2004(H9N2)) NA gene, complete cds.: GenBank AY790307.1
Influenza A virus (A/Thailand/2(SP-33)/2004(H5N1)) neuraminidase (NA) gene, complete cds.: GenBank AYSSS152.3
Influenza A virus (A/swine/Binh Doung/0216/2010(H1N2)) NA gene for neuraminidase, complete cds.: GenBank AB628082.1
Influenza A virus (A/chicken/Jalgaon/8824/2006(H5N1)) neuraminidase (NA) gene, complete cds.: GenBank DQ887063.1
Influenza A virus SC35M M2 and M1 genes, complete cds.: GenBank DQ266100.1
Influenza virus type/Leningrad/134/47/57 (H2N2) M1 and M2 RNA, complete cds.: GenBank M81582.1
Influenza A virus SC35M M2 and M1 genes, complete cds.: GenBank DQ266100.1
Influenza A virus (A/Tochigi/2/2010(H1N1)) M2, M1 genes for matrix protein 2, matrix protein 1, complete cds.: GenBank AB704481.1

Hepatitis B core and surface antigens, the sequence of which is indicated or can be derived, for example, from the following sequences:
Hepatitis B virus strain HBV248 precore protein and core protein genes, complete cds.: GenBank KP857118.1
Hepatitis B virus strain HBV401 precore protein and core protein genes, complete cds.: GenBank KP857113.1
Hepatitis B virus strain HBV403 precore protein and core protein genes, complete cds.: GenBank KP857068.1
Hepatitis B virus S gene for hepatitis B surface antigen, partial cds. isolate B0503327(PTK): GenBank AB466596.1

The process (2) of inoculation by spraying or soaking recombinant baculovirus into pyralid moths eggs, and process of expression a recombinant protein of the present invention, which one are selected from the group consisting of Meal moth (Pyralis farinalis), Stored nut moth (Paralipsa gularis), Indianmeal moth (Plodia interpunctella), Tea Tabby (Aglossa dimidiatus), Rice moth (Corcyra cephalonica), Mediterranean flour moth (Ephestia kuehniella), Cacao moth (Ephestia elutella), and Almond moth (Cadra cautella) and, belonging to the pyralid moths (which are insect pests of stored foods). And the chorionic layer and wax layer of eggshell are removed and the surface-disinfected pyralid moths eggs and, after being packaged in a sterile container with a ventilation structure, it is stored at low temperature condition of 4˜8° C., and it supplied. Preferably, in the species Mediterranean flour moth, pyralid moths eggs (chorionic layer and wax layer of eggshell are removed) are stored in a sterilized container with a ventilation structure at low temperature condition, and it supplied.

And the recombinant baculovirus (derived from Autographa californica multinuclear polyhedrosis virus; AcMNPV) is sprayed onto the above-mentioned pyralid moths eggs or, preferably, inoculated the recombinant baculovirus derived from AcMNPV by use soaking method the above-mentioned pyralid moths eggs in the liquid containing the recombinant baculovirus derived from AcMNPV. Also, incubate pyralid moths eggs (inoculated the recombinant baculovirus derived from AcMNPV) for a time sufficient to express at least one recombinant protein.

Finally, cultured for a time sufficient to express the recombinant protein and, collect above-mentioned pyralid moths eggs comprising at least one recombinant protein and, store frozen at −20° C. condition. At above-mentioned −20° C. frozen condition, pyralid moths eggs can be stored for up to about one year, it is possible to supply to all over the world through the process of refrigeration transportation using aircraft and ship.

The process (3) of producing products by harvesting and purifying recombinant proteins from pyralid moths eggs of the present invention, the above-mentioned pyralid moths eggs comprising at least one recombinant protein and, store frozen at −20° C. condition, and it supplied. And one or more recombinant proteins are included and crushes the above-mentioned pyralid moths eggs stored in the frozen. Finally, from crushed liquids of above-mentioned pyralid moths eggs comprising at least one recombinant protein is isolated and purified to produce the final recombinant protein product.

The present invention described above, in the technical field of the present invention, various modifications and applications can be made by the ordinarily skilled in the art, the scope of the technical idea according to the present invention should be defined by the following claims.

1. A pyralid moth egg belonging to any one species selected from a group consisting of: Meal moth (Pyralis farinalis), Stored nut moth (Paralipsa gularis), Indianmeal moth (Plodia interpunctella), Tea Tabby (Aglossa dimidiatus), Rice moth (Corcyra cephalonica), Mediterranean flour moth (Ephestia kuehniella), Cacao moth (Ephestia elutella), and Almond moth (Cadra cautella) and, the pyralid moth egg comprising at least one of recombinant baculovirus and bacmids comprising a nucleic acid sequence encoding a recombinant protein derived from Autographa californica multinuclear polyhedrosis virus (AcMNPV).

2. The pyralid moth egg according to claim 1, wherein the recombinant protein is selected from a group consisting of: subunit monomeric vaccine, subunit multimeric vaccine, virus like particle, therapeutic protein, antibody, enzyme, cytokine, blood clotting factor, anticoagulant, receptor, hormone, diagnostic protein reagents, and green fluorescent protein (GFP) and, the pyralid moth egg comprising the recombinant protein which is not an endogenously produced protein by an egg.

3. A method, comprising:
providing a pyralid moth egg belonging to any one species selected from a group consisting of: Meal moth (Pyralis farinalis), Stored nut moth (Paralipsa gularis), Indianmeal moth (Plodia interpunctella), Tea Tabby (Aglossa dimidiatus), Rice moth (Corcyra cephalonica), Mediterranean flour moth (Ephestia kuehniella), Cacao moth (Ephestia elutella), and Almond moth (Cadra cautella), wherein the pyralid moth egg is provided by:
providing the pyralid moth egg as surrounded by an eggshell having a multilayer structure of a vitelline membrane, a waxy layer and a chorionic layer;
treating the pyralid moth egg with a nontoxic chemical to remove the chorionic layer from the pyralid moth egg surrounded by the eggshell;
treating the pyralid moth egg with a nontoxic composition to remove the waxy layer from the pyralid moth egg; and
collecting the pyralid moth egg having the chorionic layer and the wax layer of eggshell removed and a disinfected surface.

4. A method for producing at least one recombinant protein, comprising:
providing a pyralid moth egg having a chorionic layer and a wax layer of an eggshell of the pyralid moth egg removed and a disinfected surface, the pyralid moth egg belonging to any one species selected from a group consisting of: Meal moth (Pyralis farinalis), Stored nut moth (Paralipsa gularis), Indianmeal moth (Plodia interpunctella), Tea Tabby (Aglossa dimidiatus), Rice moth (Corcyra cephalonica), Mediterranean flour moth (Ephestia kuehniella), Cacao moth (Ephestia elutella), and Almond moth (Cadra cautella);
inoculating recombinant baculovirus by spraying a liquid containing the recombinant baculovirus onto the pyralid moth egg or soaking the pyralid moth egg in the liquid containing the recombinant baculovirus;
incubating the pyralid moth egg as inoculated for a predetermined amount of time to express the at least one recombinant protein;
collecting the pyralid moth egg as incubated comprising the at least one recombinant protein;
harvesting the at least one recombinant protein from the pyralid moth egg; and
producing a purified recombinant protein by purifying the at least one recombinant protein.

5. The method of claim 3, wherein the nontoxic chemical is 3% sodium hypochlorite.

6. The method of claim 3, wherein the nontoxic composition is 90% D-limonene+5% Cocamide DEA+5% ethoxylated alcohol.

7. The method of claim 4, wherein the recombinant baculovirus is derived from Autographa californica multinuclear polyhedrosis virus (AcMNPV).
